(19) United States
(12) Patent Application Publication
(10) Pub. No.: US 2023/0190695 A1
(43) Pub. Date: Jun. 22, 2023

(54) TRIBUTYRIN SUPPLEMENTATION PROVIDES BENEFITS FOR MENTAL WELLNESS, IMMUNE HEALTH AND FAT METABOLISM

(71) Applicant: PHARMAVITE LLC, West Hills, CA (US)

(72) Inventors: Vandana Sharma, San Diego, CA (US); Susan Hazels Mitmesser, Agoura hills, CA (US)

(73) Assignee: PHARMAVITE LLC, West Hills, CA (US)

(21) Appl. No.: 17/971,542

(22) Filed: Oct. 21, 2022

Related U.S. Application Data
(60) Provisional application No. 63/271,093, filed on Oct. 22, 2021.

(51) Int. Cl. A61K 31/22; A61K 9/00; A61P 37/02; A61P 25/22

(52) U.S. Cl. CPC A61K 31/22; A61K 9/0053; A61P 25/22; A61P 37/02

(57) ABSTRACT

A method of improving mood, improving coping with stress, improving metabolism, and/or improving immune system function including administering of an amount of tributyrin to or by a subject sufficient to release at least a portion of butyrate from the administered amount in the small intestine of the subject to improve a mood, improve a coping with stress, improve a metabolism and/or improve an immune system function of the subject. A method of improving mood, improving coping with stress, improving metabolism, and/or improving immune system function including administering of an amount of tributyrin to or by a subject sufficient to release a first portion of butyrate from the administered amount in the small intestine of the subject and a second portion of butyrate in the colon to improve mood, improve coping with stress, improve a metabolism and/or improve an immune system function of the subject. A dietary supplement composition comprising an amount of tributyrin sufficient to release at least a portion of butyrate in the small intestine and a second portion in colon.

### CROSS-REFERENCE TO RELATED APPLICATION SECTION

The present Application claims the benefit of U.S. Provisional Pat. Application No. 63/271,093 entitled “Tributyrin Supplementation Provides Benefits for Mental Wellness, Immune Health and Fat Metabolism,” filed Oct. 22, 2021, the contents of which are incorporated in this disclosure by reference in their entirety.

### FIELD OF THE INVENTION

Tributyrin administration.

### BACKGROUND OF THE INVENTION

Our gastro-intestinal system has trillions of microbes, collectively called gut microbiota/microbiome. It has been well established that diet impacts gut microbial composition and metabolite production, which in turn influences overall health of the host. Microbial metabolites generated in human gut are absorbed and play a crucial role in most biological processes such as immunity, metabolism, brain function, etc. Gut-brain axis promotes a bidirectional communication between emotional and cognitive centers and gastrointestinal system. 70% of our immune system resides in our gut, therefore gut microbes play an important role in regulating immune processes.

Microbial metabolites such as short chain fatty acids, tryptophan, serotonin, indole, etc. contribute to overall health. Tryptophan is an amino acid utilized to synthesize proteins. Intestinal bacteria can directly utilize tryptophan to produce many immunologically important metabolites such as indole, indolic acid derivatives and tryptamines in the gut. Many bacterial species can convert tryptophan into indole and indole derivatives through an enzyme, tryptophanase. Indole acts as an intercellular signaling molecule within the gut microbial ecosystem and has been demonstrated to interact with the gut epithelium. Cooperation between gut microbes possessing the enzymes tryptophan monooxygenase and indole acetamide hydrolase enables the conversion of indole to indole-3-acetic acid, which has been demonstrated to play a role in regulating the intestinal immunity and is thought to be involved in bacterial signaling and colonization of the healthy flora in the gut. The role of indole and its derivatives is an emerging area of research and there is now some evidence of their beneficial effects. Therapeutic administration of oral indole propionic acid was protective in a murine model of colitis suggesting anti-inflammatory role) and neuroprotective effects in rats. Indole acetic acid (IAA) alleviated high fat diet-induced hepatotoxicity in mice. Tryptophan is also a precursor for serotonin synthesis, a neurotransmitter which affects mood and melatonin which induces sleep.

Tributyrin when consumed orally releases butyrate. Butyrate is a beneficial short chain fatty acid primarily produced by gut microbiota and is a major energy source for colonic cells. It also provides anti-inflammatory effects.

Diet plays an important role in modulating gut microbiome. Enrichment of beneficial gut microbes can provide health benefits. For example - Gut bacteria Faecalibacterium prausnitzii is a butyrate-producing strain, which exerts strong anti-inflammatory activity in the intestinal environment, mainly linked with the stimulation of regulatory T-cells Akkermansia muciniphilla, Parabacteroides distasonis and Parabacteroides goldsteinii improves metabolic parameters.

### BRIEF DESCRIPTION OF THE DRAWINGS

FIG. 1 shows Average concentration (mM) ± stdev (n = 3) of tributyrin (left) and butyrate (right) during passage through the upper gastrointestinal tract (GIT) under fed or fasted conditions in a gut simulation model for the encapsulated tributyrin product. Data are representative for samples collected during passage in the stomach (ST) and small intestine (SI). Statistically significant differences as compared to the preceding time point were indicated with * (p < 0.05).

FIG. 2 shows the butyrate levels in both the proximal colon (PC) compartment and the distal colon (DC) compartment following of administration of different doses of tributyrin in an in vitro gut simulation model;

FIG. 3 shows Reciprocal Simpson diversity index for the microbial diversity in the lumen of the proximal colon (PC) and distal colon (DC) upon treatment with high dose of tributyrin averaged over the replicates at different time points during the study, i.e., at the end of the control period (Cn=9) and at the end of the treatment period (TR; n=9);

FIG. 4 shows the ratio of Firmicutes and Bacteroidetes in the distal colon for low and high dose tributyrin treatment compared to a control;

FIG. 5 shows the presence of Akkermansia muciniphila in the proximal colon (PC) and distal colon (DC) following administration of different doses of tributyrin in an in vitro gut simulation model with abundance values presented as absolute abundance of shot gun sequencing data corrected by flow cytometry counts. Abundance of A. muciniphilla in Control is hardly visible on the graph, whereas both low and high tributyrin treatments lead to a considerable increase in its abundance;

FIG. 6 shows the production of Faecalibacterium Prausnitzii in the proximal colon (PC) and distal colon (DC) following administration of different doses of tributyrin in an in vitro gut simulation model with abundance values presented as absolute abundance of shot gun sequencing data corrected by flow cytometry counts. Abundance of F. prausnitzii in control is hardly visible on the graph, whereas both low and high tributyrin treatments lead to a considerable increase in its abundance;

FIG. 7 shows the bacterial count for certain Parabacteroides spp. following administration of different doses of tributyrin in an in vitro gut simulation model with abundance values presented as absolute abundance of shot gun sequencing data corrected by flow cytometry counts;

FIG. 8 shows immobility time per minute in a force swim test for groups of mice with and without tributyrin administration;

FIG. 9 shows corticosterone levels after forced swim test in groups of mice with and without tributyrin administration;

FIG. 10 shows percentage change in fat mass from the baseline in groups of mice treated with tributyrin for nearly 8 weeks compared to mice that did not receive tributyrin with body composition measured using nuclear magnetic resonance (NMR);

FIG. 11 shows change in the lean:fat ratio in groups of mice treated with tributyrin for nearly 8 weeks compared to mice that did not receive tributyrin, with body composition measured using NMR;

FIG. 12 shows various cytokine concentrations in serum for mice at the end of 8 weeks of tributyrin administration compared to mice that did not receive tributyrin;

FIG. 13 shows various cytokine concentrations in serum for mice at the end of 8 weeks of tributyrin administration compared to mice that did not receive tributyrin; and

FIG. 14 shows the levels of tryptophan and indole-3-acetic acid in fecal samples of mice treated with tributyrin for nearly 8 weeks compared to mice that did not receive tributyrin.

### DETAILED DESCRIPTION

For purposes of the following description, the use of “or” means “and/or” unless specifically stated otherwise, even though “and/or” may be explicitly used in certain instances.

As used herein, “including,” “containing” and like terms are understood in the context of this application to be synonymous with “comprising” and are therefore open-ended and do not exclude the presence of additional undescribed or unrecited elements, materials, ingredients or method steps.

As used herein, “consisting of” is understood in the context of this application to exclude the presence of any unspecified element, ingredient or method step.

As used herein, “consisting essentially of” is understood in the context of this application to include the specified element(s), material(s), ingredient(s) or method step(s) and those that do not materially affect the basic and novel characteristic(s) of what is being described.

As used herein, “subject” or “individual” means animals, including mammals, including humans, a canine, a feline, a bovine, an equine, a porcine, a primate, and/or a rodent. Also, as used herein, “administering” an amount (e.g., a dose) of a composition may be done by the subject himself/herself or another subject (e.g., a medical professional, a caretaker, a family member). The composition may be provided to the subject or the administrator for the subject along with instructions for administration of the composition (e.g., written instructions on a label of a container containing the composition).

A composition is described. The composition includes, consists essentially of or consists of an amount of tributyrin or a tributyrin derivative sufficient to release at least a portion of butyrate in the small intestine or an amount of tributyrin sufficient to release a first portion of butyrate in the small intestine and a second portion of butyrate in the colon. The composition may be in the form of a dietary or nutritional supplement that is added to a diet (supplements a normal human diet). Butyrate is a short chain fatty acid (SCFA) and is a major energy source for colon. Most butyrate is endogenously produced in the gut by resident microbiota and absorbed by colonic cells. It is believed that only a minor fraction of this naturally produced butyrate reaches systemic circulation. Tributyrin is a triglyceride, essentially 3 butyrate molecules attached to a glycerol chain. When consumed orally, it is acted upon by esterases thereby releasing butyrate. Administration of an amount of tributyrin or tributyrin derivative to or by a subject sufficient to release at least a portion of butyrate from the administered amount in the small intestine of the subject provides butyrate that can be absorbed and be available in systemic circulation. The systemically-circulated butyrate is available to reach other organs including the brain via blood and could impart various health benefits. Brain health benefits from administration of tributyrin include improving mood through alleviation of stress, anxiety and/or depression. Administration of an amount of tributyrin or a tributyrin derivative to or by a subject sufficient to release a first portion of butyrate from the administered amount in the small intestine and a second portion of butyrate in the colon provides butyrate to the systemic circulation for brain health benefits as well as butyrate to the colon to modulate gut microbiome and microbial metabolites of the subject, which also have a potential to provide brain health benefits. A method of improving mood comprising administering of an amount of tributyrin or tributyrin derivative to or by a subject sufficient to release at least a portion of butyrate from the administered amount in the small intestine of the subject to improve a mood of the subject or an amount of tributyrin or tributyrin derivative sufficient to release a first portion of butyrate in the small intestine and a second portion of butyrate in the colon is also described. In addition to mood improvement or other brain health benefits, other health benefits to administration of tributyrin to a subject include improved body metabolism and improved immune system function. A method of improving metabolism comprising administering of an amount of tributyrin or tributyrin derivative to or by a subject sufficient to release at least a portion of butyrate from the administered amount in the small intestine of the subject or an amount of tributyrin or tributyrin derivative sufficient to release a first portion of butyrate in the small intestine and a second portion of butyrate in the colon is also described to improve a metabolism of the subject is further described. A method of improving immune system function comprising administering of an amount of tributyrin or tributyrin derivative to or by a subject sufficient to release at least a portion of butyrate from the administered amount in the small intestine of the subject or an amount of tributyrin or tributyrin derivative sufficient to release a first portion of butyrate in the small intestine and a second portion of butyrate in the colon is also described to improve an immune system function of the subject.

A composition as a supplement (a nutritional or dietary supplement) includes an amount of tributyrin to or by a subject sufficient to release at least a portion of butyrate from the administered amount in the small intestine of the subject. For an adult human subject, a representative amount is 100 mg/day to 2000 mg/day, such as 300 mg/day to 2000 mg/day, such as 300 mg/day to 1000 mg/day, such as 500 mg/day to 800 mg/day as a daily dosage. As used herein, a “daily dosage” of an amount of tributyrin is an amount of tributyrin consumed in one day, either all at once (one setting) or through multiple settings throughout a day. The dosage level may be administered as a single dose administered to or by a subject, or through multiple administrations (multiple doses) that achieve, for example, a daily dosage level over the course of a day. The dosage level may also be a total amount of tributyrin administered for multiple times per week, weekly, bi-weekly, or monthly administration divided by the number of days between administration, wherein the dose administers an amount of tributyrin in a dosage level described above on a per day average. The dosage level described above relates to tributyrin, a compound providing three butyrate molecules. Alternatively, or additionally, a tributyrin derivative may be used as a source of butyrate. Where the tributyrin derivative comprises less than three butyrate molecules (e.g., a butyrate di-ester), the dosage level of the derivative should be increased.

The composition described herein may be in the form of a dry powder, such as in a tablet or capsule, or liquid form or a powder that can be mixed to in a liquid to form a dispersion or suspension in the liquid. The compositions may also tie in the form of a soft gel, gummy, a suppository, foam enema, liquid enema, or the like. The compositions may be formulated in such a manner as to be administered orally or rectally. The compositions of the invention may include a pharmaceutically acceptable carrier or diluent to form a tablet, capsule, solution, dispersion, emulsion, microemulsion, suspension, syrup, elixir or the like such that the materials may be swallowed or ingested.

The composition may include other active ingredients in combination with the amount of tributyrin or tributyrin derivative. An active ingredient in this sense is an ingredient that provides a beneficial effect on the individual consuming the composition and/or one or more other ingredient in the composition. Representative active ingredients include but are not limited to one or more probiotics, such as but not limited to a Lactobacillus stain (e.g., a Lactobacillus rhamnosus, a Lactobacillus fermentum) and/or a Bifidobacterium strain (e.g., a Bifidobacterium bifidum, a Bifidobacterium lactis, a Bifidobacterium longum). A representative daily dosage of a probiotic is an amount of a probiotic of at least 0.5 billion colony forming units (cfu), such as at least 1 billion cfu, such as at least 2 billion cfu, such as at least 5 billion cfu, such as at least 10 billion cfu, and such as at least 15 billion cfu. The composition may alternatively or additionally contain one or more other active ingredients, including but not limited to, one or more prebiotics (e.g., pectin (e.g., apple pectin), beta-glucan (e.g. oats or barley beta-glucan, xylooligosaccharides (XOS), inulin); one or more vitamins (e.g., vitamin A (e.g., beta-carotene), vitamin B6 (pyridoxine), vitamin B9 (folate), vitamin B12 (cobalamin), vitamin C, vitamin E, vitamin K); one or more minerals (e.g., zinc, magnesium, copper, iron) and/or one or more herbs (e.g., ginseng, chamomile, ginger, bacopa, ashwagandha, St. John’s wort, turmeric).

### 1. Effect of Tributyrin on Human Gut Microbiome in an in Vitro Model

In vitro models are useful to assess metabolic capacity of gut microbiota, which is often not possible in an in vivo system because of the constant microbiome-host interaction and absorption of microbial metabolites by the host.

### 1. Upper Gastrointestinal Tract (GIT) Simulation

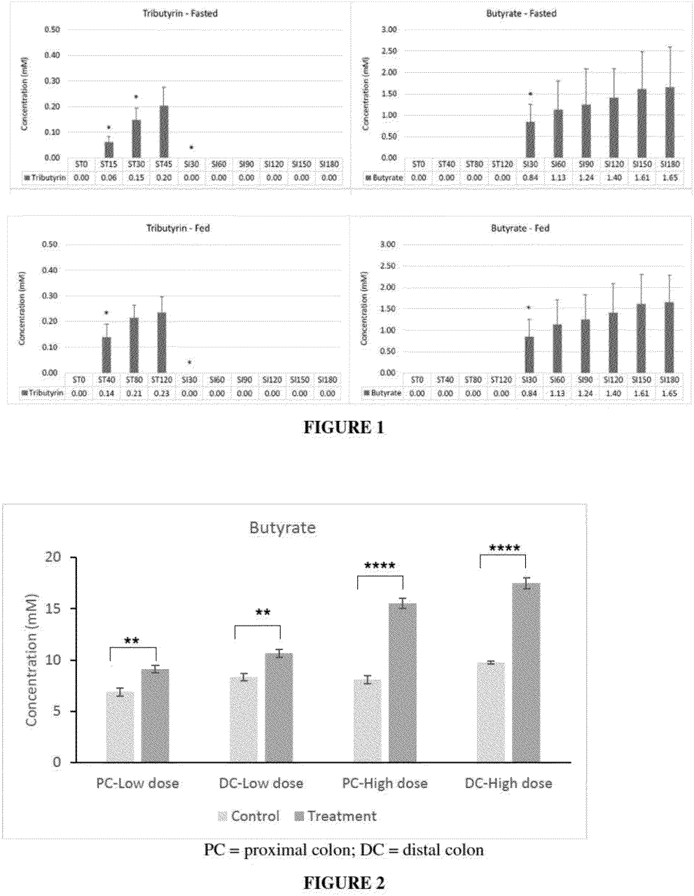

Stability of an encapsulated form of tributyrin during the passage through the simulated human upper gastrointestinal tract (GIT) under both fed and fasted conditions was assessed using an in vitro human gut simulator system that mimics the physiological conditions representative of a human GIT. FIG. 1 graphically summarizes the results of tributyrin and butyrate release in the upper GIT. During upper gastrointestinal transit, it was observed that the capsule containing tributyrin granules completely disintegrated during the first 10 minutes of stomach (ST) incubation and there was a gradual release of tributyrin from the granules under both fed and fasted conditions. While further dissolution was observed during small intestinal (SI) transit, the released tributyrin was immediately degraded by the present esterases to form butyrate. An average of 1.65 mM butyrate was detected at the end of the small intestinal incubation under both fed and fasted conditions. Considering that tributyrin contains three butyrate molecules (corresponding to 87.4% of the substrate), the observed butyrate concentrations at the end of the small intestine (small intestine for 180 minutes (SI180) in FIG. 1) corresponded to a release of 41.0% butyrate, which implies that 59.0% equivalent butyrate in the form of tributyrin would reach the colon. Moreover, part of the granules were still intact at the end of the small intestinal incubation, indicating that more tributyrin could be released when progressing to the colonic region.

### 2. Colonic Simulation

The impact of two doses of tributyrin on gut microbiota composition and microbial metabolites was studied in an in vitro gut simulation model using combined microbiota of 10 healthy adult human donors (5 males, 5 females, 30-60 years old, BMI<30). The retention times and pH ranges were optimized in order to obtain results that are representative for a full GIT simulation. A stabilization period was followed by a two-week control period and three weeks of treatment, where 59% of 300 mg/day or 59% of 1000 mg/day was administered daily (based on release profile described above). Samples were collected at the end of control period and treatment period. Both doses were performed in biological triplicate to account for biological variability.

### Butyrate Production

Initiation of the treatment with tributyrin immediately resulted in significantly enhanced butyrate levels in both the proximal colon (PC) and the distal colon (DC) compartments. As illustrated in FIG. 2, the strongest effects were observed upon supplementation of tributyrin at the highest dose tested, i.e., an average increase of 7.4 mM (or + 91.9% as compared to the control period) and 7.7 mM (or + 78.8%) in the proximal and distal colon respectively, reaching significantly higher butyrate levels as compared to treatment with the low dose of tributyrin. There was a clear dosage effect.

### Microbial Metabolites - Tryptophan and Indole Derivatives

1. While tryptophan levels remained unaffected upon treatment with the lower tributyrin dose in the proximal colon, supplementation with the higher dose resulted in significantly increased tryptophan levels (Table 1).

2. The decarboxylation pathway converts tryptophan to tryptamine, which acts as a trace neurotransmitter. Tryptamine can be further converted to indole-acetaldehyde, a tryptophan metabolite linked to the degradative pathway, the indole pathways. Indole-3-acetic acid levels significantly increased upon treatment with the lower tributyrin dose in both the proximal and distal colon. Indole-3-lactic acid levels, on the other hand, significantly increased in both colon regions upon supplementation of either tributyrin dose. Since it has been demonstrated to play an anti-inflammatory role in the intestinal epithelial cells, increased levels are seen as beneficial to the host.

3. Indole-3-aldehyde and indole-3-ethanol both promote epithelial barrier function. Treatment with tributyrin (at the high and low dose) decreased indole-3-aldehyde levels in the distal colon, whereas the higher tributyrin dose significantly increased indole-3-ethanol levels in both colon regions.

4. Finally, indole-3-propionic acid, mainly functions as a potent neuroprotective antioxidant and demonstrates anti-inflammatory properties. Indole-3-propionic acid levels significantly increased across all replicates upon supplementation with both doses of tributyrin in the distal colon.

These results show that tributyrin treatment increases several indole derivatives with potential for health benefits.

TABLE 1

Effect of the low and high dose of tributyrin on select metabolites in the proximal (PC) and distal colon (DC), averaged over the different replicates. Statistically significant differences relative to the control period are indicated in bold; p< 0.05. <LOD = Below limit of detection

| Microbial Metabolites | Proximal Colon | | | | Distal Colon | | | |
|---|---|---|---|---|---|---|---|---|
| | Low Dose | | High Dose | | Low Dose | | High Dose | |
| | Control | Treatment | Control | Treatment | Control | Treatment | Control | Treatment |
| Tryptophan | 0.91 | 0.908 | 0.819 | 1.337 | 0.122 | 0.116 | 0.111 | 0.095 |
| Serotonin | < LOD | < LOD | < LOD | < LOD | < LOD | < LOD | < LOD | < LOD |
| 5-Hydroxyindoleacetic acid | 0.01 | 0.01 | 0.01 | 0.008 | 0.001 | 0.001 | 0.001 | 0.001 |
| Melatonin | < LOD | < LOD | < LOD | < LOD | < LOD | < LOD | < LOD | < LOD |
| Tryptamine | 0.126 | 0.118 | 0.12 | 0.105 | 0.242 | 0.205 | 0.224 | 0.175 |
| Indole | < LOD | < LOD | < LOD | < LOD | < LOD | < LOD | < LOD | < LOD |
| Indole-3-acetic acid | 1.042 | 1.497 | 0.889 | 0.916 | 0.77 | 1.356 | 0.468 | 0.644 |
| Skatole | < LOD | < LOD | < LOD | < LOD | < LOD | < LOD | < LOD | < LOD |
| Indole-3-lactic acid | 0.483 | 0.631 | 0.344 | 0.52 | 0.024 | 0.04 | 0.005 | 0.021 |
| Indole-3-aldehyde | 0.064 | 0.06 | 0.059 | 0.058 | 0.028 | 0.017 | 0.026 | 0.018 |
| Indole-3-ethanol | 0.233 | 0.266 | 0.18 | 0.243 | 0.309 | 0.284 | 0.194 | 0.271 |
| Indole-3-acrylic acid | < LOD | < LOD | < LOD | < LOD | < LOD | < LOD | < LOD | < LOD |
| Indole-3-propionic acid | 0.008 | 0.008 | 0.012 | 0.01 | 0.366 | 0.847 | 0.201 | 0.544 |
| 1-Acetyl-3-indolecarboxyaldehyde | < LOD | < LOD | < LOD | < LOD | < LOD | < LOD | < LOD | < LOD |

### Microbial Composition

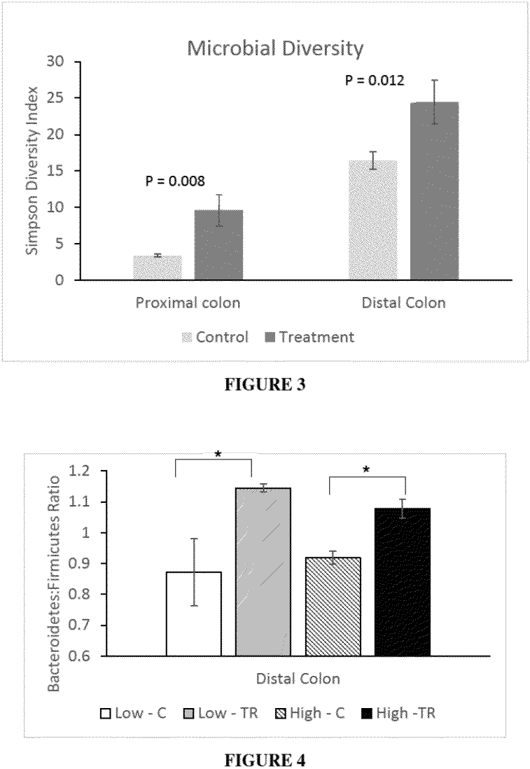

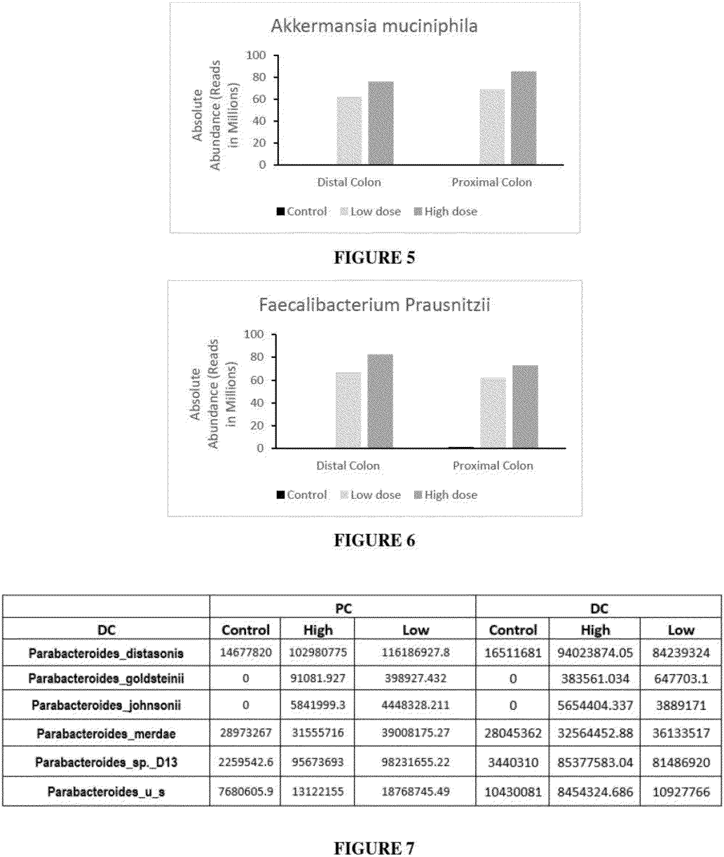

Tributyrin significantly modulated the microbial structure at different phylogenetic levels and was seen independently of the dose and colonic compartment.
There was an increase in gut microbial diversity with both low and high dose tributyrin treatment as indicated by higher Simpson Diversity Index as illustrated in FIG. 3.
Firmicutes and Bacteroidetes are most abundant phyla in human gut. Higher ratio of Bacteroidetes:Firmicutes is associated with lean phenotype. Tributyrin treatment increases this ratio as illustrated in FIG. 4 and therefore may provide metabolic health benefit.
At species level, importantly, Akkermansia muciniphila and Faecalibacterium prausnitzii were enriched in tributyrin-treated compartments.
Akkermansia muciniphila is an acetate and propionate-producing, mucin-degrading bacteria. Its presence in the gut is associated with health benefits. An inverse relationship- between colonization of Akkermansia muciniphila and inflammatory conditions or obesity have been shown. FIG. 5 shows the enrichment of Akkermansia muciniphila in the PC and DC following low- and high-dose tributyrin treatment.
Faecalibacterium prausnitzii is a butyrate-producing strain, which exerts strong anti-inflammatory activity in the intestinal environment, mainly linked with the stimulation of regulatory T-cells. As shown in FIG. 6, Faecalibacterium Prausnitzii was specifically stimulated upon supplementation of tributyrin at dose tested.
Tannerellaceae levels increased in the proximal colon upon supplementation of tributyrin. As shown in FIG. 7, the increase in Tannerellaceae levels in the proximal colon was attributed to specific stimulation of Parabacteroides spp. such as Parabacteroides merdae ATCC 43184, a bacterial strain which has been shown to promote anti-seizure effects. Parabacteroides distasonis and Parabacteroides goldsteinii have been shown to provide anti-obesity health benefits. Both Parabacteroides distasonis and Parabacteroides goldsteinii were stimulated in the proximal colon and distal colon for both low and high doses of tributyrin.

### 3. Immunomodulation

An in vitro Caco-2/THP1 (colonic cells/macrophages) co-culture model was used to assess the effect of microbial metabolites on proinflammatory or ant-inflammatory cytokines and chemokines which serve as markers for immunomodulation.

In general, low dose tributyrin treatment showed some mild immunomodulatory differences in the proximal and distal colon. In contrast, high dose tributyrin treatment showed beneficial immunomodulatory properties in the proximal colon, while stronger reduction of inflammatory markers were observed in the distal colon.

More specifically, in the proximal colon, low dose tributyrin treatment decreased the secretion of the chemokines CXCL10 and MCP-1. In contrast, high dose tributyrin treatment increased the secretion of the anti-inflammatory cytokine IL-10; while the secretion of the pro-inflammatory cytokine TNF-α and of the chemokine CXCL10 decreased. In the distal colon, secretion of the pro-inflammatory cytokine TNF-α and chemokines CXCL10 and IL-8 tended to slightly increase. In contrast, high dose tributyrin treatment showed a reduction in all cytokines and chemokines including several pro-inflammatory markers analyzed as shown in Table 2.

TABLE 2

Cell experiment results for the average of the treatment suspensions normalized to the average of the blank control suspensions. Values close to 1 indicate no change from Control, values below 1 indicate treatment lower than Control and values above 1 are indicative of treatment values higher than Control. PC = proximal colon; DC = distal colon.

| | NF-kB | IL6 | IL10 | IL-1B | TNFa | CXCL10 | MCP1 | IL8 |
|---|---|---|---|---|---|---|---|---|
| PC-Low | 0.99 | 1.01 | 1.06 | 1.08 | 1.06 | 0.79 | 0.85 | 1.3 |
| PC-High | 1.06 | 1.31 | 1.13 | 1.08 | 0.77 | 0.6 | 0.97 | 1.03 |
| DC-Low | 1.05 | 1.13 | 1.03 | 1.07 | 1.17 | 1.1 | 1.05 | 1.27 |
| DC-High | 0.66 | 0.63 | 0.53 | 0.32 | 0.3 | 0.38 | 0.38 | 0.73 |

The in vitro study of tributyrin on the human gut microbiome showed that administration of tributyrin could provide the following benefits:
Brain health (mood) benefits like alleviation of stress, anxiety and depression due to: (i) an increase in butyrate which plays an important role in gut-brain axis; (ii) an increase in tryptophan which is a precursor to serotonin and melatonin (serotonin regulates mood and melatonin induces sleep) and is metabolized into indole derivatives; (iii) an increase in levels of several indolic derivatives, including indole metabolites such as indole-3-acetic acid, indole-3-propionic acid, indole-3-lactic acid and indole-3-ethanol that are believed to provide brain health (mood) benefits and indole-3-acetic acid is a neuroprotective antioxidant; and (iv) an increase in Faecalibacterium prausnitzii, which is a butyrate-producing strain and may provide a brain health benefit.
Immune health benefits due to (i) an increase in butyrate levels with butyrate known to induce regulatory T cells (Treg) differentiation and controlling inflammation; (ii) an increase in Faecalibacterium prausnitzii, a butyrate-producing strain that exerts strong anti-inflammatory activity in the intestinal environment by stimulation of regulatory T-cells; and (iii) high dose tributyrin treatment led to a decrease in several proinflammatory markers in the distal colon (DC) and an increase in anti-inflammatory marker like IL-10 in the proximal colon (PC).
Metabolic benefits due to: (i) an increase in Akkermansia muciniphila associated with lean phenotype; (ii) an increase in indole metabolites such as indole-3-acetic acid have been shown to alleviate high fat diet induced hepatotoxicity in mice, which was with the reduction in insulin resistance and lipid metabolism; (iii) an increase in Parabacteroides distasonis and Parabacteroides goldsteinii that may provide metabolic anti-obesity health benefits such as reduction in fat mass; and (iv) an increase in Bacteroidetes:Firmicutes Ratio is also associated with lean phenotype.

### Physiological Benefit of Tributyrin On Mental Wellness (Preclinical Trial)

Considering that tributyrin showed several beneficial properties in an in vitro model, an in vivo mouse model was used to validate physiological benefits of tributyrin supplementation. Experiments were conducted to determine the effects of tributyrin supplementation on brain health, especially mood (in relation to stress induced anxiety and depression), fat mass and immune markers.

### Method

A study was conducted to evaluate tributyrin on stress and mood-related behaviors in female and male C57BL/6J mice. The animals were given immediate ad libitum access to water and standard rodent chow and were acclimated to the facility for 7 days. On study day -3, fat and muscle content was measured using nuclear magnetic resonance spectroscopy (NMR: MiniSpec NMR, Bruker LF50 Body Composition Analyzer). On study day 0, the animals were weighed, blood and fecal samples (1-2 pellets/animal) were collected, and the animals were randomized into their specific treatment group. Daily oral gavage of 4 distinct groups commenced as follows:
Treatment 1: daily PBS vehicle + no acute stress before behavioral measures
Treatment 2: daily PBS vehicle + acute stress before behavioral measures
Treatment 3: daily low-dose tributyrin (60 mg/kg/d) + acute stress before behavioral measures
Treatment 4: daily high-dose tributyrin (200 mg/kg/d) + acute stress before behavioral measures

After 7 weeks of intervention, blood and fecal samples were collected on study day 49, and fecal collection was repeated on study days 50 and 52. On study day 52, animals assigned to the acute stress groups were acutely restrained for two hours while unstressed animals were housed singly for 140 minutes in fresh cages. Following the two-hour stress period, stressed animals were released into their cage (without bedding) and had a 20-min grooming break. All animals were then tested in the forced swim test (FST). A retro-orbital blood sample was collected within 15 to 30 minutes after the FST, and fecal pellets were collected (for corticosterone measurements). On study day 53, the animals were weighed, and body composition by NMR was performed a second time. The study was terminated on study day 56, blood was collected and necropsies were performed. Serum cytokine levels were evaluated in terminal serum.

### Results

### Behavior

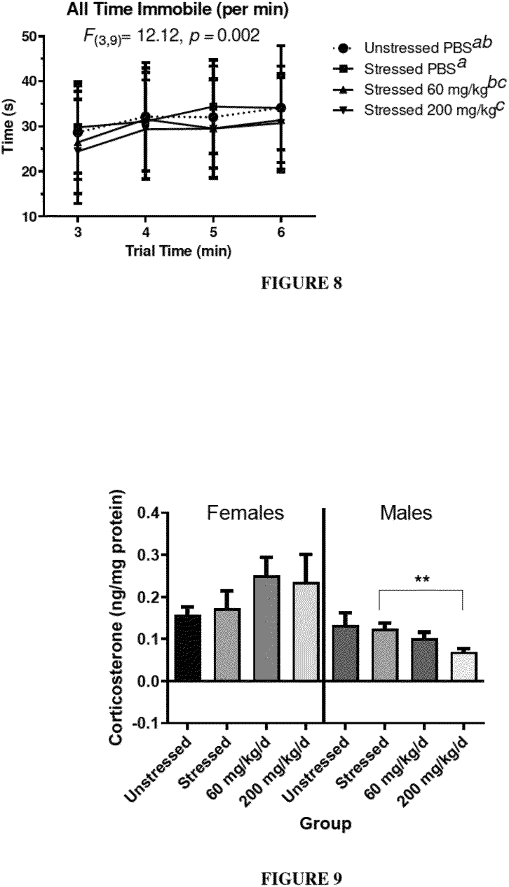

Forced Swim Test (FST) is a test centered on a rodent’s response to unescapable stress. Results of this test have been interpreted as a measure of susceptibility to negative mood. It is commonly used to measure the effectiveness of antidepressants. Forced swim tests were performed on study day 52 for each cohort of mice. Mice treated with tributyrin exhibited reduced immobility (i.e., increased positive coping skills) in the FST when analyzed by 1-way RMANOVA on a per-minute basis. As illustrated in FIG. 8, in combined (female + male) groups, reductions in time immobile in the FST were statistically significant by 1-way RMANOVA [F_{(3,9)} = 12.12, p = 0.002] for mice treated with 60 or 200 mg/kg/d tributyrin as compared to the stressed untreated control group and for mice treated at 200 mg/kg/d versus the unstressed control group. Similarly, 1-way RMANOVA detected a main effect of treatment [F_{(7,21)} = 6.10, p < 0.001] when all 8 groups were analyzed, with Tukey’s post hoc test detecting that 60 mg/kg in females and 200 mg/kg in males resulted in reduced immobility in comparison to their sex-matched, untreated, stressed control groups.

### Corticosterone

Cortisol or corticosterone are important mediators of the stress system. The corticosteroid hormones operate in concert with catecholamines and other transmitters. Insufficient corticosteroid control leads to aggravated stress reactions. Alternatively, if adaptation to stress fails, circulating corticosteroid levels remain elevated for a prolonged period of time. As illustrated in FIG. 9, male mice treated with 200 mg/kg/d tributyrin had a statistical reduction in fecal corticosterone levels after FST as compared to the stressed vehicle control group when inter cohort variability was accounted for in 2-way ANOVA.

Reduction in corticosterone and results of FST shows that tributyrin treatment increases positive coping skills, reduces depression and helps cope with stress.

### Fat Mass

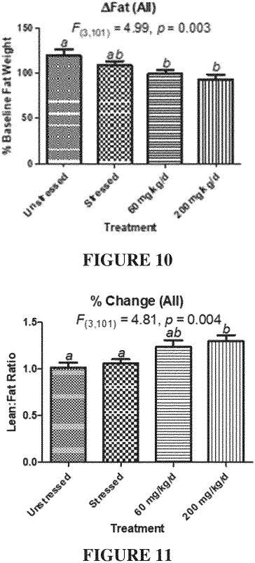

Animal body weight change did not differ statistically across groups for female or male or all mice. However, body composition analysis indicated that tributyrin administration reduced fat composition. When considering all mice (male and female), final fat as a percentage of baseline was statistically reduced in groups treated with 60 or 200 mg/kg/d tributyrin as compared to the combined unstressed untreated control groups [F_{(3,101)} = 4.99, p = 0.003] as shown in FIG. 10. Consequently, Lean:Fat ratio was significantly increased in treated mice as compared to untreated control as shown in FIG. 11. A decrease in fat mass and an increase in lean:fat ratio shows a benefit to body composition.

### Immune Markers

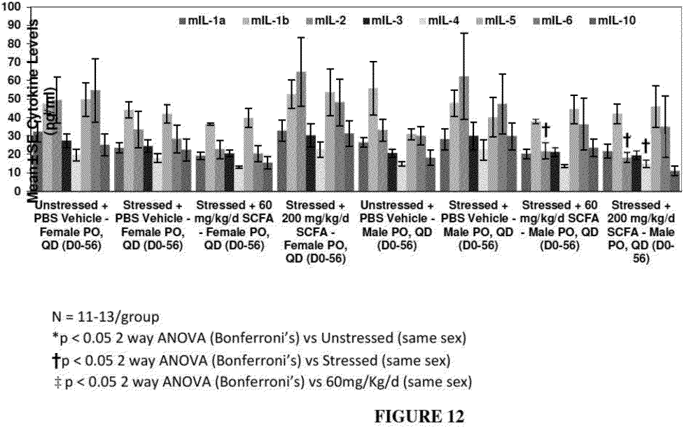

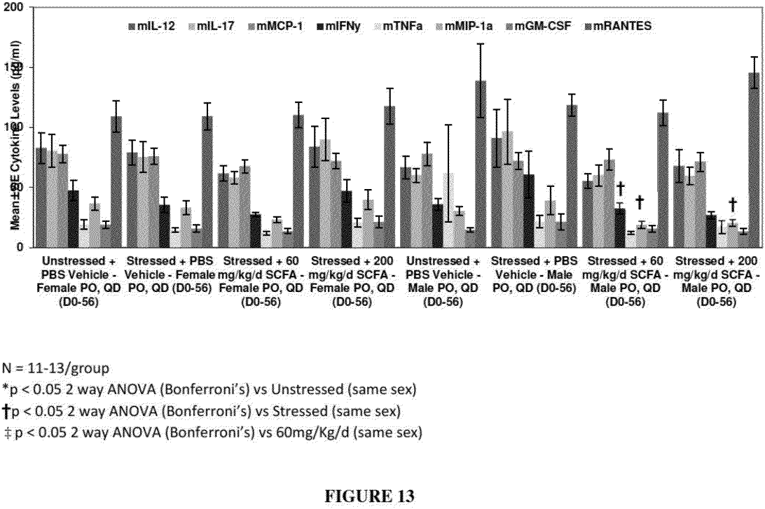

Initial analysis of cytokine concentrations in terminal serum showed that the levels of interleukins IL-2 with 60 mg/kg/d tributyrin administration and IL-4 with 60 or 200 mg/kg/d tributyrin administration reduced significantly as compared to stressed mice by the treatment in male mice (see FIG. 12). Moreover, IFN_{γ} was significantly reduced in male mice treated with 60 mg/kg/d tributyrin, and MIP-1a was significantly reduced in male mice treated with 60 or 200 mg/kg/d tributyrin as compared to the stressed vehicle control group (see FIG. 13). Reduction of pro-inflammatory markers in mice is consistent with the observations from in vitro cell culture model. These results indicate immunomodulatory potential of tributyrin and potential for an immune health benefit.

### Fecal Metabolite Analysis

Gut microbiota metabolizes ingested food and supplements in the colon thereby producing various metabolites, which have an impact on several host biological processes. These metabolites generated in the colon can be measured in the feces. A panel of metabolites were measured in feces collected from experimental mice 8 weeks post-treatment. Fecal SCFA were measured by Gas Chromatography (GC) and tryptophan metabolites were measured using Ultra High Performance Liquid Chromatography High Resolution Mass Spectrometry (UHPLC-HRMS). Comparisons were made between stressed untreated mice and tributyrin treated stressed mice.

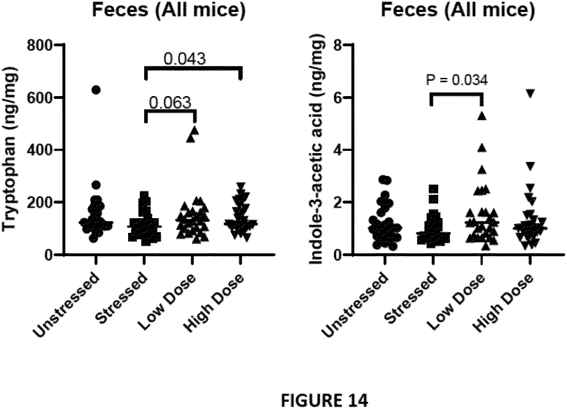

No differences were observed in fecal SCFA levels between tributyrin treated mice and untreated stressed controls. However, there were statistically significant changes in other metabolites in the feces as shown in FIG. 14:
(i) There was an increase in tryptophan with tributyrin treatment with high dose (142.8 ± 53.4 ng/mg) as well as low dose (154.9 ± 98.7 ng/mg) as compared to untreated stressed control (114.6 ± 44.8 ng/mg), which was statistically significant for high dose (p = 0.043) and showed a trend for low dose (p = 0.063). Tryptophan is an essential amino acid. It is involved in various biological processes including being a precursor for serotonin, which is a key element in enteric nervous system.
(ii) Indole-3-acetic acid was statistically significantly elevated in low dose treated mice (1.56 ± 1.17 ng/mg) compared to untreated stressed control (1.006 ± 0.507 ng/ml). It has been demonstrated to play an antiinflammatory role in the intestinal epithelial cells, therefore increased levels will be beneficial to the host.

Both tryptophan and indole-3-acetic acid were also elevated in the gut simulation model that mimics the physiological conditions representative of a human GIT indicating consistency between the results from the in vitro model and the in vivo model.

These beneficial effects in mice on mood, metabolism and immune parameters could be due to both butyrate in systemic circulation due to absorption in small intestines as well as butyrate in colon, modulating microbiota and microbial metabolites.

### Aspects

The following are aspects of the invention.

1. A method of improving at least one of mood, coping with stress, metabolism, and immune system function comprising administering of an amount of tributyrin or a tributyrin derivative to or by a subject sufficient to release at least a portion of butyrate from the administered amount in the small intestine of the subject to improve at least one of a mood of the subject, a coping with stress of the subject, a metabolism of the subject, and an immune system function.

2. The method of Aspect 1 wherein the method comprises improving the mood of the subject.

3. The method of Aspect 1, wherein the method comprises improving coping with stress.

4. The method of Aspect 1, wherein the method comprises improving the metabolism of the subject.

5. The method of Aspect 1, wherein the method comprises improving the immune system function of the subject.

6. The method of any of Aspects 1-5, wherein administering comprising administering tributyrin and the amount of tributyrin comprises 100 mg/day to 2000 mg/day, such as 300 mg/day to 1000 mg/day, such as such as 500 mg/day to 800 mg/day for an adult human.

7. The method of Aspect 6, wherein administering comprises administering the tributyrin as a supplement.

8. The method of any of Aspects 1-7, wherein the portion of butyrate from the administered amount of tributyrin or a tributyrin derivative is a first portion and the amount of tributyrin or a tributyrin derivative administered comprises an amount sufficient to release a second portion of butyrate in the colon.

9. A method of improving at least one of mood, coping with stress, metabolism, and immune system function comprising administering of an amount of tributyrin or tributyrin derivative to or by a subject as a daily dosage sufficient to release a first portion of butyrate from the administered amount in the small intestine of the subject and a second portion of butyrate in the colon to improve at least one of a mood of the subject, a coping with stress of the subject, a metabolism of the subject, and an immune system function.

10. The method of Aspect 9, wherein the method comprises improving the mood of the subject.

11. The method of Aspect 9, wherein the method comprises improving coping with stress.

12. The method of Aspect 9, wherein the method comprises improving the metabolism of the subject.

13. The method of Aspect 9, wherein the method comprises improving the immune system function of the subject.

14. The method of any of Aspects 9-13, wherein administering comprising administering tributyrin and the amount of tributyrin comprises 100 mg/day to 2000 mg/day, such as 300 mg/day to 1000 mg/day, such as such as 500 mg/day to 800 mg/day for an adult human.

15. The method of Aspect 14, wherein administering comprises administering the tributyrin as a supplement.

16. A dietary supplement composition comprising an amount of tributyrin or tributyrin derivative sufficient to release at least a first portion of butyrate in the small intestine and a second portion in the colon of a subject.

17. The dietary supplement composition of Aspect 16, wherein the composition comprises tributyrin and the amount of tributyrin comprises 100 mg/day to 2000 mg/day, such as 300 mg/day to 1000 mg/day, such as 500 mg/day to 800 mg/day for an adult human.

18. The dietary supplement composition of Aspect 16 or Aspect 17, wherein the tributyrin or tributyrin derivative comprises a first active ingredient and the dietary supplement further comprises a second active ingredient different than the first active ingredient.

Whereas specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims and aspects appended and any and all equivalents thereof.

What is claimed is:

1. A method of improving at least one of mood, coping with stress, metabolism, and immune system function comprising administering of an amount of tributyrin or a tributyrin derivative to or by a subject sufficient to release at least a portion of butyrate from the administered amount in the small intestine of the subject to improve at least one of a mood of the subject, a coping with stress of the subject, a metabolism of the subject, and an immune system function.

2. The method of claim 1 wherein the method comprises improving the mood of the subject.

3. The method of claim 1, wherein the method comprises improving coping with stress.

4. The method of claim 1, wherein the method comprises improving the metabolism of the subject.

5. The method of claim 1, wherein the method comprises improving the immune system function of the subject.

6. The method of claim 1, wherein administering comprising administering tributyrin and the amount of tributyrin comprises 300 mg/day to 1000 mg/day for an adult human.

7. The method of claim 6, wherein administering comprises administering the tributyrin as a supplement.

8. The method of claim 1, wherein the portion of butyrate from the administered amount of tributyrin or a tributyrin derivative is a first portion and the amount of tributyrin or a tributyrin derivative administered comprises an amount sufficient to release a second portion of butyrate in the colon.

9. A method of improving at least one of mood, coping with stress, metabolism, and immune system function and comprising administering of an amount of tributyrin or tributyrin derivative to or by a subject as a daily dosage sufficient to release a first portion of butyrate from the administered amount in the small intestine of the subject and a second portion of butyrate in the colon to improve at least one of a mood of the subject, a coping with stress of the subject, a metabolism of the subject, and an immune system function.

10. The method of claim 9, wherein the method comprises improving the mood of the subject.

11. The method of claim 9, wherein the method comprises improving coping with stress.

12. The method of claim 9, wherein the method comprises improving the metabolism of the subject.

13. The method of claim 9, wherein the method comprises improving the immune system function of the subject.

14. The method of claim 9, wherein administering comprising administering tributyrin and the amount of tributyrin comprises 100 mg/day to 1000 mg/day for an adult human.

15. The method of claim 14, wherein administering comprises administering the tributyrin as a supplement.

16. A dietary supplement composition comprising an amount of tributyrin or tributyrin derivative sufficient to release at least a first portion of butyrate in the small intestine and a second portion in the colon of a subject.

17. The dietary supplement composition of claim 16, wherein the composition comprises tributyrin and the amount of tributyrin comprises 100 mg/day to 2000 mg/day for an adult human.

18. The dietary supplement composition of claim 16, wherein the composition comprises tributyrin and the amount of tributyrin comprises 300 mg/day to 1000 mg/day for an adult human.

19. The dietary supplement composition of claim 16, wherein the tributyrin or tributyrin derivative comprises a first active ingredient and the dietary supplement further comprises a second active ingredient different than the first active ingredient.
